SEQUENCE LISTING (6 sequences)

SEQ ID NO: 1 - DNA, 21 bp, Artificial Sequence
  Description of Combined DNA/RNA Molecule Synthetic oligonucleotide
  Description of Artificial Sequence Synthetic oligonucleotide
ggguucccugaaauguuagtt

SEQ ID NO: 2 - DNA, 21 bp, Artificial Sequence
  Description of Combined DNA/RNA Molecule Synthetic oligonucleotide
  Description of Artificial Sequence Synthetic oligonucleotide
cuaacauuucagggaaccctg

SEQ ID NO: 3 - DNA, 21 bp, Artificial Sequence
  Description of Combined DNA/RNA Molecule Synthetic oligonucleotide
  Description of Artificial Sequence Synthetic oligonucleotide
ggcuugauuucaaccuugutt

SEQ ID NO: 4 - DNA, 21 bp, Artificial Sequence
  Description of Combined DNA/RNA Molecule Synthetic oligonucleotide
  Description of Artificial Sequence Synthetic oligonucleotide
acaagguugaaaucaagcctg

SEQ ID NO: 5 - DNA, 21 bp, Artificial Sequence
  Description of Combined DNA/RNA Molecule Synthetic oligonucleotide
  Description of Artificial Sequence Synthetic oligonucleotide
ggauucauaaacuugugugtt

SEQ ID NO: 6 - DNA, 21 bp, Artificial Sequence
  Description of Combined DNA/RNA Molecule Synthetic oligonucleotide
  Description of Artificial Sequence Synthetic oligonucleotide
cacacaaguuuaugaaucctg

(19) United States
(12) Patent Application Publication
(10) Pub. No.: US 2007/0015720 A1
(43) Pub. Date: Jan. 18, 2007

(54) Modulation of endogenous AICAR levels for the treatment of diabetes and obesity

(75) Inventors: Karen G. Bulock, Providence, RI (US); Mark A. Tepper, Newton, MA (US); John Y. Zhang, Wayland, MA (US); Roustem R. Nabioullin, Hollis, NH (US)

Correspondence Address: FISH & NEAVE IP GROUP;ROPES & GRAY LLP, 1251 AVENUE OF THE AMERICAS FL C3, NEW YORK, NY, 10020-1105, US

(73) Assignee: CytRx Corporation, Los Angeles, CA (US)

(21) Appl. No.: 11/347,811

(22) Filed: Feb. 3, 2006

Related U.S. Application Data
(60) Provisional application No. 60/649,942, filed on Feb. 4, 2005.

(51) Int. Cl. A61K 31/7056

(52) U.S. Cl. 514/43

(57) ABSTRACT

The invention relates to methods for treating type 2 diabetes, obesity, metabolic syndrome and conditions associated with these by administering an AICAR-monophosphate (AICAR-MP) enhancing agent that increases endogenous AICAR-MP levels in a cell. Inhibition of AICAR-formyltransferase activity (AICARFT) in a cell that regulates metabolic activity (such as fat, liver, muscle, pancreatic beta or certain brain cells) increases AICAR-monophosphate levels which in turn results in activation of the AMP-kinase (AMPK) pathway, and all of the downstream functions mediated by AMPK including increased fatty acid oxidation, enhanced glucose transport and decreased fatty acid synthesis.

### CROSS-REFERENCE TO RELATED APPLICATIONS

This application claims the benefit of U.S. Provisional App. No. 60/649,942 filed Feb. 4, 2005, the disclosure of which is incorporated by reference in its entirety.

### TECHNICAL FIELD OF THE INVENTION

The invention relates to methods for treating type 2 diabetes, obesity, metabolic syndrome and conditions associated with these by administering an AICAR-monophosphate (AICAR-MP) enhancing agent that increases endogenous AICAR-MP levels in a cell. Inhibition of AICAR-formyltransferase activity (AICARFT) in a cell that regulates metabolic activity (such as fat, liver, muscle, pancreatic beta or certain brain cells) increases AICAR-monophosphate levels which in turn results in activation of the AMP-kinase (AMPK) pathway, and all of the downstream functions mediated by AMPK including increased fatty acid oxidation, enhanced glucose transport and decreased fatty acid synthesis.

### BACKGROUND OF THE INVENTION

Adenosine nucleotides (ATP, ADP, and AMP) are the major source of chemical energy storage in mammals. Under normal physiological conditions, the ratio of ATP/ADP/AMP is tightly controlled. However, under conditions of increased metabolic demand, such as during exercise, ATP levels decrease rapidly whereas ADP and AMP levels rise. This rise in AMP and ADP levels triggers cells to increase their metabolism of fatty acids, glucose, and amino acids in order to generate more ATP by oxidative phosphorylation. One way in which cells respond to an elevation in AMP levels is to activate the AMP-kinase (AMPK) pathway which is a key pathway in the control of fuel metabolism. When AMP-kinase is activated in cell types such as muscle and liver, these cells reduce fatty acid synthesis and increase fatty acid oxidation. Thus AMPK plays a key role in energy homeostasis making it an important target for development of drugs to treat obesity and type 2 diabetes, as well as other conditions and syndromes associated with metabolism.

5′-AMP-activated protein kinase (AMPK) is a cytoplasmic serine/threonine kinase which is allosterically activated by AMP (Corton, J. M. et al. Current Biol. 4: 315-324 (1994)), and is thus very sensitive to changes in the AMP/ATP ratio as an indicator of cellular energy state. The binding of AMP to AMPK results in phosphorylation of threonine-172 of its alpha-subunit by AMPK kinase (AMPKK) and activation of AMPK (Hawley, S. A. et al., J. Biol. Chem. 271: 27879-27887 (1996)). Once activated, AMPK causes a number of downstream effects that ultimately lead to increased fuel metabolism through oxidative phosphorylation.

For instance, in the liver and adipose tissue, AMPK phosphorylates and inactivates acetyl-CoA carboxylase 1 (ACC1), a key enzyme involved in the biosynthesis of fatty acids (Hardie, D. G. et al., Eur. J. Biochem. 246:259-273 (1997); Henin et al., FASEB J. 9:541-546 (1995)). In the liver and skeletal muscle, AMPK phosphorylates and inactivates acetyl-CoA carboxylase 2 (ACC2), a second isozyme of ACC that converts acetyl-CoA to malonyl-CoA in these tissues (Hardie et al., supra). By reducing malonyl-CoA levels, AMPK activation causes an increase in the CPT-1 mediated transport of fatty acid into the mitochondria, resulting in increased fatty acid beta-oxidation. AMPK has also been shown to activate malonyl-CoA decarboxylase in skeletal muscle, further depleting malonyl-CoA (Saha et al., J. Biol. Chem. 275:24279-24284 (2000)). AMPK also inactivates hydroxymethylglutaryl-CoA (HMG-CoA) reductase (Hardie et al., supra; Henin et al. supra), which is involved in cholesterol biosynthesis.

In addition to its effects on fatty acid metabolism, AMPK activation has been shown to increase glucose transport in muscle (Winder et al., Am. J. Physiol. 277:E1-E10 (1999)); Mu et al., Mol. Cell 7:1085-1094 (2001)) and suppress gluconeogenesis in the liver (Zhou et al., J. Clin. Invest. 108:1167-1174 (2001)). There is evidence that some of the positive effects of the anti-diabetic drugs rosiglitazone and metformin are mediated through modulation of AMPK activity (Zhou et al., supra, Saha et al., Biochem. Biophys. Res. Comm. 314:580-585 (2004); Fryer et al., J. Biol. Chem. 277: 25226-25232 (2002)).

The purine nucleoside analog, 5-aminoimidazole-4-carboxamide ribonucleoside (AICAR), in its monophosphate form (“AICAR-MP”), mimics AMP to activate AMPK (Corton, J. M. et al., Eur. J. Biochem., 229, pp. 558-565 (195). Upon administration to cells, AICAR is taken up and phosphorylated by adenosine kinase to form the active AICAR-MP ribonucleotide. AICAR-MP is a naturally occurring metabolite in the de novo synthesis pathway of purine nucleotides.

As an activator of AMPK, AICAR has been used experimentally in vitro and in vivo to decipher biological effects on metabolic pathways caused by activation of the AMPK pathway. Long term treatment with high doses of AICAR was shown to reduce plasma triglyceride and free fatty acid levels as well as decrease systolic blood pressure and decrease fasting concentrations of glucose and insulin in obese Zucker (fa/fa) rats, an animal model for insulin resistance (Buhl, E. S. et al., Diabetes 51: 2199-2206 (2002)). Exogenous administration of AICAR does not interfere with intracellular purine nucleotide pools (Corton et al., 1995, supra). Unfortunately AICAR must be administered at very high concentrations due to poor bioavailability and the relatively weak ED₅₀ of 0.2-1.5 mM (in a standard kinase assay) of AICAR-MP for AMPK (Corton et al., 1995, supra.). The dosage of AICAR required to produce physiologically relevant levels of AICAR-MP in cells would not be practical as a therapeutic for humans. Thus, it would be desirable to develop a therapeutic method to elevate endogenous levels of AICAR-MP.

AICAR formyltransferase (AICARFT) is one of two enzyme activities on the bifunctional protein, AICAR Transformylase/IMP Cyclohydrolase (ATIC) (Rayl, E. A. et al., J. Biol. Chem. 271, pp. 2225-2233 (1996)) which catalyzes the penultimate and final steps in the de novo synthesis of inosine-monophosphate (IMP). The reaction catalyzed by AICARFT involves the transfer of a formyl group from N¹⁰-formyl tetrahydrofolic acid to AICAR-MP producing 5-formyl-AICAR-MP (FAICAR-MP) (FIG. 1). Because of the key importance of purine biosynthesis in cellular proliferation, ATIC has become a target of interest for development of anticancer and anti-inflammatory drugs. In fact, the anti-inflammatory effects of low dose methotrexate are thought by some to be due to inhibition of AICARFT (Cronstein, B. N. et al., J. Clin. Investigation, 92, pp. 2675-2682 (1993)).

Treatment with the DHFR inhibitor methotrexate, or the NSAID sulfasalazine, both drugs which have been shown to inhibit AICARFT, causes a three-fold increase of AICAR-monophosphate in splenocytes of mice in the murine pouch model of inflammation (Cronstein et al., supra, Gadangi, P. et al., J. Immunol., 156, pp. 1937-1941 (1996)). Patients treated with low dose methotrexate for psoriasis also show a statistically significant increase in urinary excretion of aminoimidazole carboxamide (AICA) on the day of dosing (Baggot, J. E. et al., Archives of Dermatology, 135, pp. 813-817 (1999)) suggesting that methotrexate treatment can elevate endogenous AICAR levels.

To date, however, the lack of a specific inhibitor of AICARFT has made it difficult to determine the direct effects of AICARFT inhibition. In particular, there are no reports showing the effects of inhibition of AICARFT in metabolic tissues such as muscle, liver, or adipose.

### SUMMARY OF THE INVENTION

The present invention helps fill the needs discussed above by providing methods for increasing endogenous AICAR-monophosphate (AICAR-MP) concentrations in a mammalian cell or tissue to a level that activates AMP-kinase (AMPK) (FIG. 2). The invention thus addresses the need for safe and effective treatments of obesity, type 2 diabetes, insulin resistance, metabolic syndrome and syndromes, conditions and/or complications associated with any of the foregoing.

In one embodiment, the invention provides a method by which inhibition of the enzyme AICARFT causes an increase of the AICAR-MP concentration inside a cell to levels, e.g., that mimic the effects of exogenous AICAR treatment, and thereby activate AMPK activity. This embodiment of the invention provides methods using specific inhibitors of the enzyme AICAR formyltransferase (AICARFT) as a means to build up endogenous AICAR-MP to levels capable of activating AMPK in mammals (FIG. 2) and elicits metabolic effects that can be used to treat obesity, type 2 diabetes, insulin resistance, metabolic syndrome and syndromes, conditions and/or complications associated with any of the foregoing.

The present invention thus provides methods for treating obesity, type 2 diabetes, metabolic syndrome and/or conditions, syndromes or complications associated with these, the methods comprising the step of administering to an animal (e.g., a mammal, including a human) in need thereof an agent that inhibits AICARFT activity in an amount sufficient to increase AICAR-MP concentrations in the cell.

In one embodiment, administration of an inhibitory agent results in at least 5% inhibition of AICARFT activity over a 24 hour period. Preferably, about 5% to about 10%, more preferably about 10% to about 20% inhibition, even more preferably about 20% to about 50% or more inhibition of AICARFT activity is achieved over a 24 hour period.

The present invention also provides a method for increasing endogenous AICAR-MP levels in a metabolic tissue or cell comprising the step of administering to an animal in need thereof an inhibitor of AICARFT in an amount sufficient to increase AMP kinase activity. In a preferred embodiment, the metabolic cell or tissue is selected from the group consisting of muscle, liver and adipose, pancreatic beta cells and cells, such as neurons, and regions of the brain that control metabolic homeostasis.

The present invention also provides a method for increasing the oxidation of fatty acids in a metabolic cell or tissue comprising the step of administering an inhibitor of AICARFT in an amount sufficient to inhibit AICARFT enzyme activity and thereby stimulate fatty acid oxidation. In a preferred embodiment, the metabolic cell or tissue is selected from the group consisting of muscle, liver, adipose and pancreatic beta cells, and cells from regions of the brain that control metabolic homeostasis.

The present invention also provides a method for increasing glucose uptake in a metabolic cell or tissue comprising the step of administering an inhibitor of AICARFT in an amount sufficient to inhibit AICARFT enzyme activity and thereby stimulate glucose uptake. In a preferred embodiment, the metabolic cell or tissue is selected from the group consisting of muscle, liver, adipose and pancreatic beta cells, and cells from regions of the brain that control metabolic homeostasis.

The present invention also provides a method for decreasing fatty acid synthesis in a metabolic cell or tissue comprising the step of administering an inhibitor of AICARFT in an amount sufficient to inhibit AICARFT enzyme activity and thereby inhibit fatty acid synthesis. In a preferred embodiment, the metabolic cell or tissue is selected from the group consisting of muscle, liver, adipose and pancreatic beta cells, and cells from regions of the brain that control metabolic homeostasis.

In another aspect, the invention provides a method for identifying an agent useful for treating obesity, type 2 diabetes, insulin resistance, metabolic syndrome and syndromes, conditions and/or complications associated with any of the foregoing, comprising the step of screening one or more putative agents in a metabolic tissue or cell for effects on fatty acid beta-oxidation, fatty acid synthesis, glucose uptake and AMP kinase activation through AICARFT inhibition. An agent that increases fatty acid beta-oxidation, glucose uptake or AMP kinase activation, or decreases fatty acid synthesis through AICARFT inhibition is identified as being useful for said treatments.

The invention also provides a method for increasing AMP kinase activity in a metabolic tissue or cell comprising the step of administering an inhibitor of AICARFT in an amount sufficient to mimic the effects of exogenous AICAR treatment.

Other features and advantages of the invention will be apparent from the following detailed description, and from the claims.

### BRIEF DESCRIPTION OF THE DRAWINGS

FIG. 1. Reactions Catalyzed by Bifunctional ATIC. AICAR and N¹⁰-formyltetrahydrofolate (N¹⁰-F-FH4) are converted to formyl-AICAR (FAICAR) and tetrahydrofolate (FH₄) by the AICAR formyltransferase (AICARFT) domain of the bifunctional enzyme ATIC. FAICAR is then cyclized to form inosine monophosphate (IMP) by the IMP cyclohydrolase (IMPCH) domain.

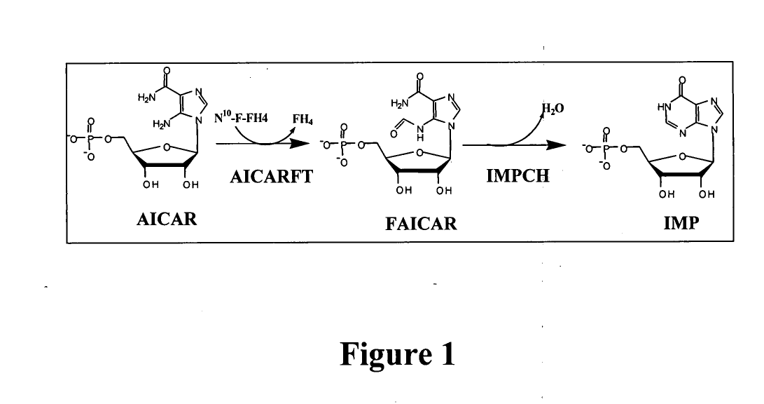

FIG. 2. Modulation of endogenous AICAR-MP Levels as a Means to Treat Metabolic Diseases Such as Diabetes and Obesity. Increasing endogenous AICAR-MP levels by selective inhibition of AICARFT represents a novel strategy for treatment of metabolic diseases. Inhibition of AICARFT would cause a build-up of AICAR-MP to levels that would result in activation of AMPK and subsequent downstream effects, including decreased fatty acid synthesis and increased beta-oxidation of long-chain acyl-CoA.

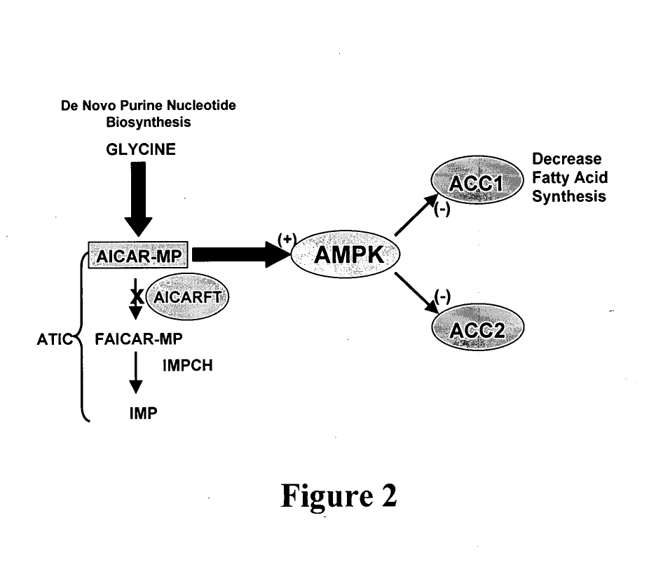

FIG. 3. An AICARFT Inhibitor Decreases Lipid Accumulation during Adipocyte Differentiation. Adipocyte differentiation was carried out as described (Example 1). 3T3L1 preadipocytes were differentiated in the presence or absence of an AICARFT inhibitor CPD-01. After differentiation, treated and control cells were stained with Oil Red O to detect the level of accumulated lipid (as an indicator of differentiation). The stain was extracted and quantified by determining absorbance at 492 nm (“Oil Red Absorbance 492 nm”). Cell samples were: 3T3L1 preadipocytes (Undiff), 3T3L1 preadipocytes that were differentiated in the presence of PBS vehicle (PBS), 3T3L1 preadipocytes that were differentiated in the presence of a DMSO control (DMSO) and 3T3L1 preadipocytes that were differentiated in the presence of AICARFT inhibitor CPD-01 (25 μM).

FIG. 4. An AICARFT Inhibitor Increases Fatty Acid Beta-Oxidation in C2C12 Muscle Cells. Beta-oxidation of ¹⁴C-palmitic acid was performed as described in Example 2. Fold-increase in counts per minute (CPM) (¹⁴CO₂ released) of treated over control (PBS) cells was calculated and averaged for three independent experiments (error bars shown). Samples were: PBS—control untreated C2C12 cells; AICAR 1 mM—C2C12 cells treated with 1 mM AICAR for 2 hours; CPD-01 compound treatments (12 μM, 25 μM, 50 μM, and 100 μM)—C2C12 cells treated with AICARFT inhibitor CPD-01 at the indicated concentration for 2 hours.

FIG. 5. An AICARFT Inhibitor Increases Fatty Acid Beta-Oxidation in Differentiated Adipocytes. Adipocyte differentiation was carried out as described in Example 1. Beta-oxidation assays were performed as described in Example 2. Fold-increase in counts per minute (CPM) (¹⁴CO₂ released) of treated over control (DMSO) cells is shown from a single experiment. Samples were: DMSO—control untreated differentiated 3T3 μl adipocytes; CPD01 (25 μM, 50 μM)—differentiated 3T3L1 adipocytes treated with 25 μM or 50 μM AICARFT inhibitor CPD-01 for 2 hours.

FIG. 6. An AICARFT Inhibitor Increases Glucose Uptake in Differentiated 3T3L1 Adipocytes. The glucose uptake assay was performed as described in Example 3. PBS—control untreated differentiated adipocytes; insulin—differentiated adipocytes treated for 1 hour with 100 nM insulin; [-] μM inhibitor—differentiated adipocytes treated with 6 μM, 12 μM, or 25 μM AICARFT inhibitor CPD-01 for 24 hours before the glucose uptake assay was performed. Fold-increase (counts per minute of ³H-2-deoxy-D-glucose; proportional to glucose uptake) of inhibitor treated over control (PBS) cells is shown from a single experiment with samples run in duplicate (error bars shown).

FIG. 7. An AICARFT Inhibitor Decreases Fatty Acid Synthesis in HepG2 Cells. Fatty acid synthesis assays were performed as described in Example 4. Results are shown in counts per minute (CPM) of incorporated ¹⁴C-acetate after treatment with control or AICARFT inhibitor in single experiments with samples run in duplicate (error bars shown): A) HepG2 cells were treated with DMSO (Control) or an AICARFT inhibitor (30 μM) (CPD-01) overnight and fatty acid synthesis assays then performed. B) In a separate experiment, HepG2 cells were treated overnight with AICAR (1 mM) or Control (PBS or DMSO) and fatty acid synthesis assays then performed.

FIG. 8. Knockdown of ATIC with siRNA Enhances the Phosphorylation of AMPK in C2C12 Muscle Cells.

(A) The effect of siRNA knockdown of AICARFT in C2C12 cells on phospho-AMPK levels was determined as described in Example 5. Lane 1, control siRNA, lane 2, AICARFT siRNA ID 79903, lane 3, AICARFT siRNA ID 79999, lane 4, AICARFT siRNA ID 80093. (B) Gene silencing at the mRNA level was monitored by quantitative RT-PCR 48 hours post transfection using DNA primers specific for AICARFT (Example 5). Shown here are PCR products using primers specific for AICARFT and 18s rRNA (QuantumRNA™ Classic 18S Internal Standard, Ambion, Austin, Tex.) was included as an internal control. Control siRNA, 4 μl (lane 1) and 8 μl (lane 2) PCR products, AICARFT siRNA (ID 79903), 4 μl (lane 3) and 8 μl (lane 4) PCR products, AICARFT siRNA (ID 79999), 4 μl (lane 5) and 8 μl (lane 6) PCR products, AICARFT siRNA (ID 80093), 4 μl (lane 7) and 8 μl (lane 8) PCR products.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well known in the art. Generally, nomenclatures used in connection with, and techniques of, cell and tissue culture, molecular biology, immunology, microbiology, genetics and protein and nucleic acid chemistry described herein are those well known and commonly used in the art. The methods and techniques of the present invention are generally performed according to conventional methods well known in the art and as described in various general and more specific references that are cited and discussed throughout the present specification unless otherwise indicated. The nomenclatures used in connection with, and the laboratory procedures and techniques of, molecular and cellular biology, biochemistry and medicinal and pharmaceutical chemistry described herein are those well known and commonly used in the art. Standard techniques are used for chemical syntheses, chemical analyses, pharmaceutical preparation, formulation, and delivery, and treatment of patients.

All publications, patents and other references mentioned herein are incorporated by reference.

The following terms, unless otherwise indicated, shall be understood to have the following meanings:

As used herein, the terms “AICARFT inhibitor” or “AICARFT inhibitory agent” refer interchangeably to an agent that decreases the observed rate of (or otherwise interferes with) a reaction catalyzed by AICAR formyl transferase (AICARFT). Preferred AICARFT inhibitors exhibit specificity in binding to AICARFT. For example, preferred AICARFT inhibitors have IC50s for AICARFT at concentrations lower than for DHFR. More preferred AICARFT inhibitors have IC50s for AICARFT at concentrations lower than for GARFT.

As used herein, the term “activity” refers, with respect to a reaction or process, to the observed rate or progression of the reaction or process. An activity modulator may, however, also encompasses and agent having an affect on, e.g., affinity constant, inhibition constant, binding rate, stability or half-life, bioavailability, in vivo uptake, metabolic and/or excretion rates, breakdown products, immunogenicity, toxicity, and the like.

As used herein, the terms “AICAR-MP enriching, enhancing or stimulating agent” refer interchangeably to an agent that increases the level of AICAR monophosphate in a cell. An AICAR-MP enhancing agent may increase the rate of formation or may increase the stability of AICAR-MP. An AICAR-MP enhancing agent may also or may alternatively inhibit the metabolism of AICAR-MP.

As used herein, the term “obesity” refers to a condition in which excess accumulation of adipose tissue, typically 20-30% above ideal body weight, is present. Alternatively, the term “obesity” refers to a body-mass index (BMI) over 30 kg/m².

As used herein, the term “type 2 diabetes” refers to a condition characterized by high blood glucose levels caused by lack of sufficient insulin and/or inability to use insulin efficiently (insulin resistance).

As used herein, the term “condition or syndrome associated with insulin resistance” refers to the inability of a mammal to use insulin efficiently and related effects including but not limited to; high blood pressure, obesity and glucose intolerance.

The term “metabolic syndrome” as used herein refers to a multiplex risk factor for cardiovascular disease including some or all of the following components:abdominal obesity, atherogenic dyslipidemia, raised blood pressure, insulin resistance with or without glucose intolerance, proinflammatory state, prothrombotic state (definition proposed by the Third Report of the National Cholesterol Education Program (NCEP) Expert Panel on Detection, Evaluation, and Treatment of High Blood Cholesterol in Adults (Adult Treatment Panel III)).

As used herein, the term “agent” refers to a molecule, compound, composition or change in a physical property that produces an observable effect.

As used herein, the term “animal” refers to a mammal, including a human. The term may also encompass cells isolated from the animal and cultured in vitro.

As used herein, the term “patient” refers to an animal in need of treatment and includes human and veterinary subjects.

As used herein, the term “metabolic cell or tissue” refers to a cell that participates in metabolic homeostasis, including but not limited to fat, muscle, liver, and pancreatic beta cells, and regions of the brain or neurons that control metabolic homeostasis.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction is at least about 10⁻⁷ M (e.g., at least about 10⁻⁸ M or 10⁻⁹ M).

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof, domains may also include distinct, non-contiguous regions of a biomolecule. Examples of protein domains include, but are not limited to, an extracellular Ig domain, a transmembrane domain, and a cytoplasmic domain.

As used herein, the term “molecule” means any compound, including, but not limited to, a small molecule, peptide, protein, sugar, nucleotide, nucleic acid, lipid, etc., and such a compound can be natural or synthetic.

As used herein the phrase “therapeutically effective amount” means an amount of a molecule of the invention, such that a subject shows an increase in AMPK mediated metabolism, which may include loss of excess body weight, increased insulin sensitivity and glucose tolerance, after being treated under the selected administration regime (e.g., the selected dosage levels and times of treatment). The term “treating” is defined as administering to a subject (e.g., a mammal; a cell in culture), a therapeutically-effective amount of a compound of the invention, to prevent the occurrence of or to control or eliminate symptoms associated with a condition, disease or disorder associated with metabolic syndrome, obesity, insulin resistance or type 2 diabetes. A subject is preferably a human or other animal patient in need of treatment.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

Standard reference works setting forth the general principles of recombinant DNA technology known to those of skill in the art include Ausubel et al., CURRENT PROTOCOLS IN MOLECULAR BIOLOGY, John Wiley & Sons, New York (1998 and Supplements to 2001); Sambrook et al., MOLECULAR CLONING: A LABORATORY MANUAL, 2d Ed., Cold Spring Harbor Laboratory Press, Plainview, N.Y. (1989); Kaufman et al., Eds., HANDBOOK OF MOLECULAR AND CELLULAR METHODS IN BIOLOGY AND MEDICINE, CRC Press, Boca Raton (1995); McPherson, Ed., DIRECTED MUTAGENESIS: A PRACTICAL APPROACH, IRL Press, Oxford (1991). Standard reference works setting forth the general principles of medical physiology and pharmacology known to those of skill in the art include: Harrison's PRINCIPLES OF INTERNAL MEDICINE, 14th Ed., (Anthony S. Fauci et al., editors), McGraw-Hill Companies, Inc., 1998. Standard reference works setting forth the general principles of protein biochemistry known to those of skill in the art include: CURRENT PROTOCOLS IN PROTEIN SCIENCE, John Wiley & Sons, New York (2004). A reference work setting forth the general principles of RNA interference is: RNAi: A GUIDE TO GENE SILENCING, Gregory J. Hannon ed. Cold Spring Harbor Laboratory Press, Cold Spring Harbor, N.Y. (2003).

Throughout this specification and claims, the word “comprise” or variations such as “comprises” or “comprising”, will be understood to imply the inclusion of a stated integer or group of integers but not the exclusion of any other integer or group of integers.

Methods and Agents for Modulating Endogenous AICAR-MP Levels

One may increase intracellular (endogenous) AICAR-MP concentrations by increasing its synthesis, inhibiting its metabolism, or both. Because AICAR-MP is the substrate for AICARFT, we hypothesized that inhibition of AICARFT in metabolic tissues would cause an increase in intracellular levels of AICAR-MP, resulting in a variety of desired downstream reactions, including activation of AMPK.

This invention is based in part on the discovery that inhibition of AICARFT with known small molecule inhibitors indeed results in physiological responses similar to those observed with exogenous AICAR treatment including increased fatty acid oxidation, increased glucose uptake, and decreased fatty acid synthesis. We have also shown that knockdown of AICARFT (ATIC) with specific siRNAs induces phosphorylation of AMPK in C2C12 muscle myoblast cells.

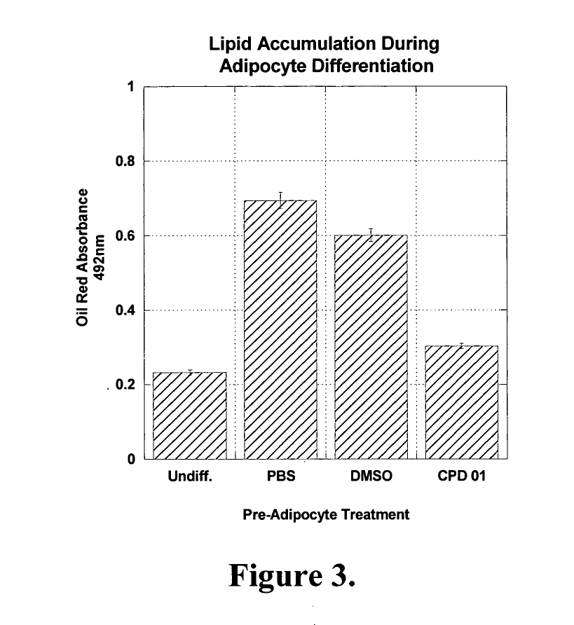

Treatment of adipocytes with AICAR inhibits adipocyte differentiation at an early stage in differentiation, although the mechanism by which this occurs is unknown (Habinowski, S. A. et al., Biophys. Res. Commun. 286, pp. 852-856 (2001)). To determine whether we could achieve a similar result with another agent, we used an AICARFT inhibitor (CPD-01) and examined its effect on lipid accumulation during the differentiation of 3T3L1 adipocytes, as described in Example 1. In this experiment, in 3T3-L1 adipocytes, lipid accumulation is proportional to the amount of oil red stain that is bound by and later extracted from adipocytes. As shown in FIG. 3, treatment with an AICARFT inhibitor (CPD-01) significantly reduced lipid accumulation in the cultured adipocytes compared to control treatments with buffer only (PBS) or solvent only (DMSO). The amount of lipid accumulation after treatment with the AICARFT inhibitor (CPD-01) was measurably higher than that for the undifferentiated pre-adipocytes assayed in parallel (Undiff.) but was clearly lower than the DMSO or PBS treated differentiated adipocytes. This result suggests that inhibition of cellular AICARFT activity in adipocytes can induce an effect similar to which is seen upon exogenous AICAR administration alone, thus supporting the hypothesis that inhibition of AICARFT can elicit biological events by causing an increase in endogenous AICAR levels.

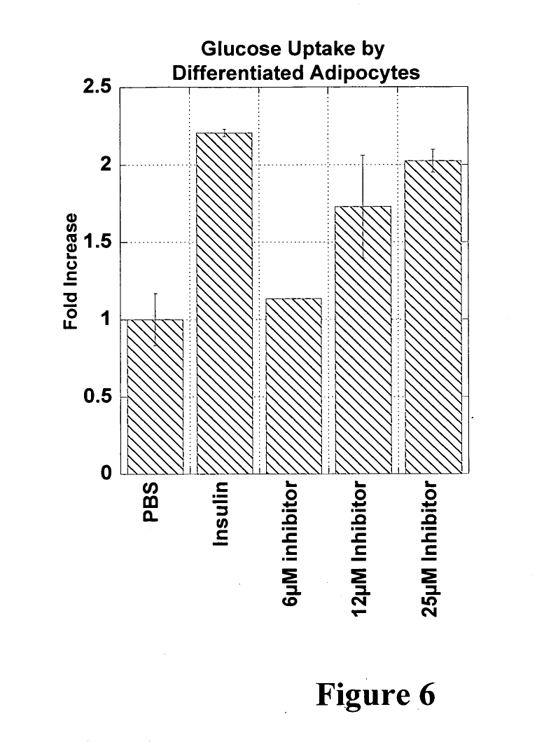

Administration of AICAR has been shown to increase non-insulin stimulated glucose uptake in 3T3L1 adipocytes (Salt, I P et al., Diabetes, 49, 1649-1656 (2000)). Increasing endogenous AICAR-MP in differentiated 3T3L1 adipocytes by treating with an AICARFT inhibitor also resulted in enhanced glucose transport into cells (Example 3). As shown in FIG. 6, differentiated adipocytes that were incubated for 24 hours with an AICARFT inhibitor at 25 μM exhibited a 2-fold increase in glucose uptake over control cells (PBS).

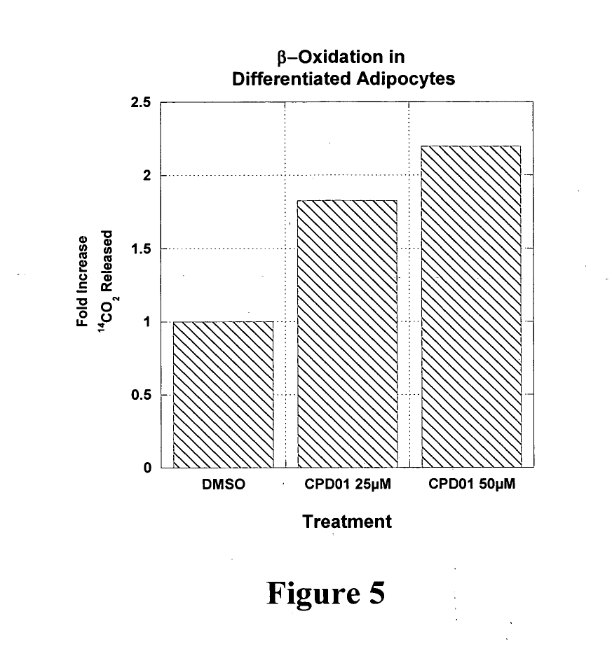

To measure the effects on fatty acid oxidation of increasing endogenous AICAR-MP levels, differentiated adipocytes were tested in a fatty acid oxidation assay, with and without treatment with the AICARFT inhibitor CPD-01 (Example 2). The results of this experiment are shown in FIG. 5 as fold-increase of radioactive CO₂ released (proportional to the extent of beta-oxidation of fatty acids) for CPD-01-treated versus DMSO (control)-treated cells. A greater than two-fold increase in beta-oxidation of palmitate was seen when differentiated 3T3-L1 adipocytes were treated with the AICARFT inhibitor. This result is consistent with the hypothesis that inhibition of AICARFT causes a buildup of AICAR-MP sufficient to activate AMPK and increase beta-oxidation.

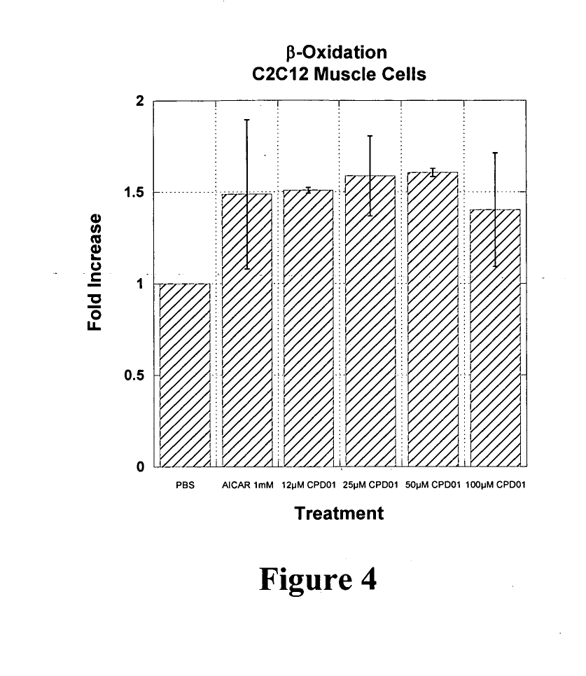

Beta-oxidation assays were similarly performed in cultured C2C12 muscle cells (Example 2), the results of which are shown in FIG. 4. Treatment of C2C12 muscle cells with an AICARFT inhibitor caused a 1.5- to 2.0-fold increase in beta-oxidation of palmitate over the control samples, which is similar to what is seen with administration of exogenous AICAR alone.

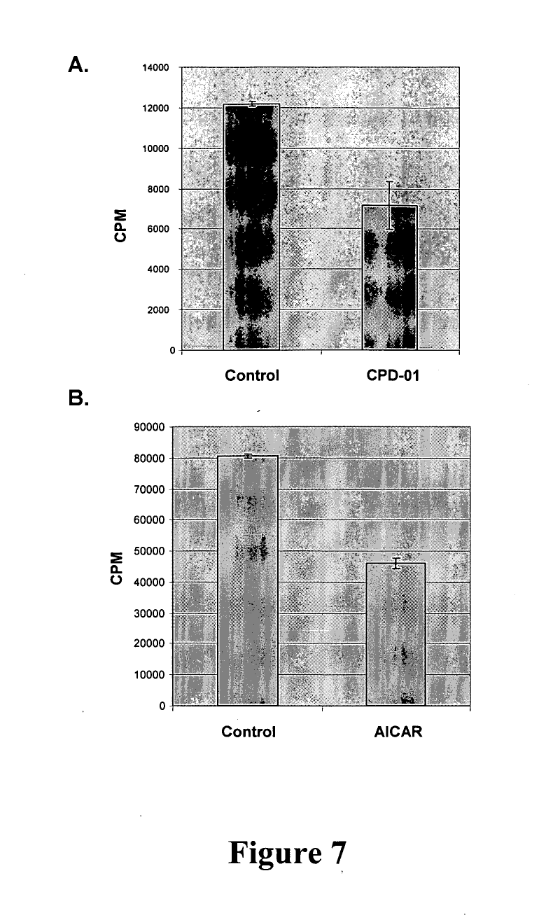

To measure the effects of an AICARFT inhibitor on fatty acid synthesis, we tested HepG2 cells in a fatty acid synthesis assay with and without treatment with AICARFT inhibitor CPD-01 (Example 4). The results are shown in FIG. 7. Treatment with AICARFT inhibitor CPD-01 resulted in a 40% decrease in incorporation of ¹⁴C-acetate into total lipids compared to control (DMSO) cells. This is a similar decrease to what is seen with exogenous AICAR treatment.

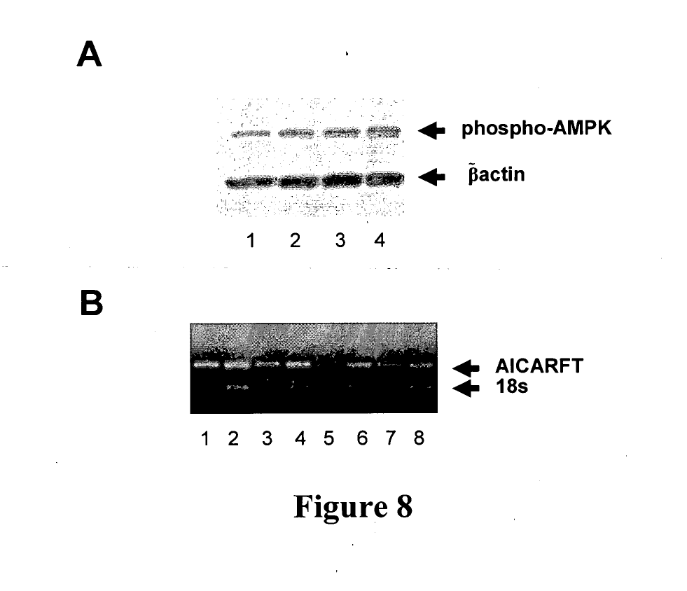

To demonstrate that specific inhibition of AICARFT results in activation of AMPK, levels of AICARFT mRNA were suppressed using siRNAs specific for this target in C2C12 muscle cells, as described in Example 5. As shown in FIG. 8, there was significant knockdown of ATIC mRNA detected by quantitative RT PCR and there was a modest corresponding increase in AMPK phosphorylation (consistent with AMPK activation).

Methods for Identifying AICAR-MP Enhancing Agents

Endogenous AICAR-MP levels may be measured directly or indirectly. AICAR-MP levels in a cell may be quantitated directly, e.g., by extracting nucleotides from cells and separating the nucleotides by HPLC as described in (Sabina et al, J. Biol. Chem. 257(17) 10178-10183 (1982)). AICAR-MP levels in a cell may be assessed indirectly, e.g., by monitoring AMPK activation, such as by quantifying phospho-AMPK levels in the cell using a commercially available antibody specific for phosphorylated AMPK, as described herein. Any method for measuring AICAR-MP levels in a cell, whether direct or indirect, may be used to identify AICAR-MP modulating agents (enhancing or inhibiting) that alter AICAR-MP levels. Such modulators can thus be identified in a straight-forward fashion. AICAR-MP modulating agents, and particularly, AICAR-MP enhancing agents, will be useful in practicing the methods of the invention

AICARFT Inhibiting Agents

AICARFT catalyzes the penultimate step in de novo purine biosynthesis. Most known AICARFT inhibitors are antifolates, used historically to modulate cellular proliferation (e.g., neoplastic treatments) and immune reactions. As discussed above, ATIC has become a target of interest for development of anticancer and anti-inflammatory drugs. While many antifolates are AICARFT inhibitors to some extent, they show a broad range of binding specificities (and hence selectivities) for AICARFT with respect to the three other major tetrahydrofolate-dependent enzymes: glycinamide ribonucleotide formyltransferase (GARFT); dihydrofolate reductase (DHFR); and thymidylate synthase (TS). Two other tetrahydrofolate-dependent enzymes relevant to specificity are serine hydroxymethyl transferase (SHMT) and methionine synthase (MS). Hence, there are a variety of known general antifolates that inhibit AICARFT activity without any significant specificity for AICARFT over other folate-dependent enzymes. Such antifolate inhibitors include, but are not limited to: piritrexim, ZD1694, lometrexol, edatrexate, trimertexate and methotrexate.

A variety of agents are known to inhibit AICARFT activity. Such agents are disclosed, e.g., in WO 00/13688 (Agouron/Pfizer); U.S. Pat. No. 6,323,210; Marsilje et al., Bioorg. Med. Chem. 11:4503 (2003); Tatlock et al. (Agouron/Pfizer), 217^{th} Am. Chem. Soc. Meeting, Anaheim, Calif. March 1999; Cheong C. G. et al., J. Biol. Chem. 279(17):18034-45 (2004); Acid Yellow 54 (Xu, L et al, J. Biol. Chem., 279(48):50555-65 (2004); Isolates from the NCI Database (Li, C et al, J. Med. Chem., 47(27):6681-90 (2004)). Any one of these AICARFT inhibitory agents may be used according to the methods of the invention. Also, as shown in FIG. 8 as a result of the experiment of Example 5, an AICARFT inhibitor may be a nucleic acid such as a small interfereing RNA (siRNA) capable of reducing expression of AICARFT by, e.g., RNA interference. Other nucleic acid-based means for reducing AICARFT in a cell are also envisioned as being effective (such as but not limited to shRNAs, microRNAs, and the like). Methods for making siRNAs and other inhibitory nucleic acid molecules are well known in the art. See, e.g., U.S. Pat. Nos. 5,898,031; 6,107,094; 6,506,559; 6,573,099; and U.S. Application Publication Nos. 2002/0160972; 2003/0108923; 2003/0153519; US2004/0053875; 2004010439; 2004/0259247; 2005/0054847; 2005/0059005; 2005/0074757; 2005/0075492; 2005/0203047; 2005/0250208; and 2005/0119202; British Patent GB2397818; European Patents, EP1214945; EP1230375; EP1144623

Preferred AICARFT inhibiting agents of the invention show increased binding specificity for AICARFT compared to other folate-dependent enzymes. Thus, certain preferred AICARFT inhibiting agents of the invention show increased binding specificity for AICARFT compared to thymidylate synthase and dihydrofolate reductase. Other preferred AICARFT inhibiting agents show increased binding specificity for AICARFT over GAR-transformylase. AICARFT inhibiting agent IC₅₀s (i.e., the concentration of agent at which 50% enzyme inhibition occurs) for AICARFT are preferably at least about 2-fold lower, more preferably at least about 3- to 5-fold lower, more preferably at least about 10-fold lower, even more preferably at least about 20- to 50-fold lower, and are most preferably 50-100-fold or more lower than the AICARFT inhibiting agent's IC₅₀ for one or more other folate-dependent enzymes.

Representative AICARFT inhibitors of the invention have IC₅₀s for AICARFT that are about 75 μM-100 μM, more preferably about 25 μM-50 μM, more preferably about 5 μM-10 μM, even more preferably about 1 μM, even more preferably about 0.5 μM or 0.1 μM, and most preferably about 50-100 nM, 25-50 nM, 5-25 nM, 1-5 nM, or less.

An AICARFT inhibiting agent may also be modified or derivatized with other molecules to increase or otherwise alter its binding specificity and/or its activity. One of skill in the art has available a variety of methods which may be used to alter the biological and pharmacological properties of an AICARFT inhibiting agent or other AICAR-MP enhancing agent to increase its utility for practicing the methods of the invention, e.g., to modulate activity (e.g., affinity constant, inhibition constant, binding rate, stability or half-life, bioavailability, in vivo uptake, metabolic and/or excretion rates, breakdown products, immunogenicity, toxicity) or to alter it in any other way that may render it more suitable for a particular application.

Other AICARFT inhibiting agents, including non-folate inhibitors, may be identified and/or verified after identification using, e.g., any known AICARFT activity assay. One such assay is described in Black et al., Analytical Biochem. 90: 397-401 (1978). AICARFT inhibiting agents identified by this or any other relevant method (e.g., computer-assisted, virtual docking or other structural modeling programs) will be useful in practicing the methods of the invention. AICARFT inhibiting agents may be used alone or in combination with other AICARFT inhibiting agents, and/or agents that are currently or that will in the future be used to treat obesity, type 2 diabetes, metabolic syndrome and their complications.

Treatment Methods

This invention is based on experiments that show for the first time that inhibition of AICARFT in a cell increases oxidation of fatty acids and glucose uptake in metabolic cells and tissues. Therefore, AICAR-MP enhancing agents, including but not limited to AICARFT inhibitors, are useful in methods for treating for treating obesity, type 2 diabetes, insulin resistance, metabolic syndrome and syndromes, conditions and/or complications associated with any of the foregoing.

Accordingly, the invention provides a method for treating obesity, type 2 diabetes or insulin resistance syndrome comprising the step of administering to an animal in need thereof an inhibitor of AICARFT in an amount sufficient to inhibit enzyme activity.

In one aspect, the present invention provides a method for treating obesity, type 2 diabetes or insulin resistance syndrome comprising the step of administering to an animal in need thereof an inhibitor of AICARFT in an amount sufficient to inhibit AICARFT enzyme activity. In one embodiment, administration of the inhibitor results in at least 5% inhibition of AICARFT activity over a 24 hour period. Preferably, administration of the inhibitor results in from about 5% to about 10% inhibition of AICARFT activity over a 24 hour period. More preferably, administration of the inhibitor results in from about 10% to about 20%, from about 20% to about 50%, or more than 50% inhibition of AICARFT activity over a 24 hour period.

The present invention also provides a method for increasing endogenous AICAR-monophosphate levels in a metabolic tissue or cell comprising the step of administering to an animal in need thereof an inhibitor of AICARFT in an amount sufficient to cause desired effects similar to those observed with exogenous AICAR administration, including but not limited to increasing AMP kinase activity and some or all of the known activities regulated by AMPK activation. In certain embodiments, the metabolic tissue or cell is one that is associated with (e.g., within or derived from) an animal in need of treatment for obesity, type 2 diabetes, insulin resistance, metabolic syndrome and syndromes, conditions and/or complications associated with any of the foregoing. In certain other embodiments, the metabolic tissue or cell excludes cancer and tumor cells and proliferating cells involved in immune response, e.g., B cells and T cells. In yet other embodiments, the metabolic tissue or cell is selected from the group consisting of muscle, liver, adipose, pancreatic beta cells and cells of the brain that control metabolic homeostasis.

The present invention also provides a method for increasing the beta-oxidation of fatty acids in a metabolizing cell or tissue comprising the step of administering an inhibitor of AICARFT in an amount sufficient to inhibit AICARFT enzyme activity. In certain embodiments, the metabolic tissue or cell is one that is associated with (e.g., within or derived from) an animal in need of treatment for obesity, type 2 diabetes, insulin resistance, metabolic syndrome and syndromes, conditions and/or complications associated with any of the foregoing. In certain other embodiments, the metabolic tissue or cell excludes cancer and tumor cells and proliferating cells involved in immune response, e.g., B cells and T cells. In yet other embodiments, the metabolic tissue or cell is selected from the group consisting of muscle, liver, adipose, pancreatic beta cells and cells of the brain that control metabolic homeostasis.

The present invention also provides a method for increasing insulin sensitivity and/or glucose uptake in a metabolic cell or tissue comprising the step of administering an inhibitor of AICARFT in an amount sufficient to inhibit AICARFT enzyme activity. In certain embodiments, the metabolic tissue or cell is one that is associated with (e.g., within or derived from) an animal in need of treatment for obesity, type 2 diabetes, insulin resistance, metabolic syndrome and syndromes, conditions and/or complications associated with any of the foregoing. In certain other embodiments, the metabolic tissue or cell excludes cancer and tumor cells and proliferating cells involved in immune response, e.g., B cells and T cells. In yet other embodiments, the metabolic tissue or cell is selected from the group consisting of muscle, liver, adipose, pancreatic beta cells and cells of the brain that control metabolic homeostasis.

The present invention also provides a method for inhibiting adipocyte differentiation and a method for suppressing lipid accumulation comprising the step of administering an inhibitor of AICARFT in an amount sufficient to inhibit AICARFT enzyme activity.

The present invention also provides a method for inhibiting fatty acid synthesis comprising the step of administering an inhibitor of AICARFT in an amount sufficient to inhibit AICARFT enzyme activity.

Any AICARFT inhibitor may be used in methods of the invention. Preferred AICARFT inhibitors for use with the methods of the invention have an inhibition constant (Ki) of 10 nanomolar or less. Other preferred AICARFT inhibitors have an inhibition constant (Ki) in the range of 25-50 nanomolar. Still other preferred AICARFT inhibitors have an inhibition constant (Ki) in the range of 50-200 nanomolar. AICARFT inhibitors having an inhibition constant (Ki) greater than 200 nanomolar are also considered useful. Preferred AICARFT inhibitors for use with the invention include those that show selectivity for AICARFT over other folate dependent metabolic or catabolic reactions. Certain preferred selective AICARFT inhibitors inhibit AICARFT at lower concentrations than they inhibit DHFR (i.e., an inhibitor which has an IC₅₀ for AICARFT that is lower than its IC₅₀ for DHFR). The IC₅₀ for AICARFT and DHFR will differ 2- to 5-fold, more preferably 5- to 10-fold, even more preferably more than 10-fold. More preferred selective AICARFT inhibitors inhibit AICARFT at lower concentrations than they inhibit GAR-transformylase. The IC₅₀ for AICARFT and GAR-transformylase will differ 2- to 5-fold, more preferably 5- to 10-fold, even more preferably more than 10-fold.

Treatment with an AICAR-MP enhancing agent, such as an AICARFT inhibitor, may be accomplished by any mode selected by the skilled worker, e.g., a treating physician. Modes of administration include but are not limited to IP, IV, oral, transdermal, and local administration into muscle or fat. Oral administration is preferred. The dose of an AICAR-MP enhancing agent, such as an AICARFT inhibitor, is chosen to be in the range of about 0.1 to about 1000 mg/kg/BID, more preferably in the range of about 0.5 to about 500 mg/kg/BID, even more preferably in the range of about 1 to about 100 mg/kg/BID, or about 10 to about 50 mg/kg/BID. Preferred doses may be determined empirically by the skilled artisan.

The present invention also provides a method for identifying an agent useful for treating obesity, type 2 diabetes, insulin resistance, metabolic syndrome and syndromes, conditions and/or complications associated with any of the foregoing, comprising the step of screening one or more putative agents in a metabolic tissue or cell for effects on fatty acid beta-oxidation, glucose uptake, fatty acid synthesis or AMP kinase activation, wherein an agent that increases fatty acid beta-oxidation, glucose uptake or AMP kinase activation of decreases fatty acid synthesis is identified as a useful agent.

The present invention also provides a method for inhibiting AICARFT in a metabolic tissue or cell comprising the step of administering an inhibitor of AICARFT in an amount sufficient to increase AMP kinase activity.

Pharmaceutical Compositions and Treatments

The AICAR-MP enhancing agents, including AICARFT inhibitors used in conjunction with this invention may be formulated into pharmaceutical compositions and administered in vivo at an effective dose to treat the particular clinical condition addressed. Administration of one or more of the pharmaceutical compositions according to this invention will be useful for regulating glucose uptake and insulin action, for reducing lipid accumulation and for treating type 2 diabetes, obesity, insulin resistance and associated conditions and syndromes and metabolic syndrome and associated conditions. Such conditions and syndromes include, but are not limited to abdominal obesity, atherogenic dyslipidemia, raised blood pressure, insulin resistance with or without glucose intolerance, proinflammatory state, prothrombotic state.

Enhancing and inhibitory agents of this invention may be administered alone or in combination with one or more therapeutic or diagnostic agents. For example, the compositions of this invention may be administered together with but not limited to, e.g., anti-inflammatory agents, anticoagulants, antithrombotics, cholesterol-lowering drugs, binding and/or stabilizing agents, cytokines, hormones and the like.

The patient to be treated may be a human or a veterinary animal.

Determination of a preferred pharmaceutical formulation and a therapeutically efficient dose regimen for a given application is within the skill of the art taking into consideration, for example, the condition and weight of the patient, the extent of desired treatment and the tolerance of the patient for the treatment.

Administration of the enhancing and inhibitory agents of this invention, including isolated and purified forms, their salts or pharmaceutically acceptable derivatives thereof, may be accomplished using any of the conventionally accepted modes of administration of agents which are used to regulate glucose uptake and insulin action, to reduce lipid accumulation and to treat obesity, type 2 diabetes, insulin resistance, metabolic syndrome and syndromes, conditions and/or complications associated with any of the foregoing.

The pharmaceutical compositions of this invention may be in a variety of forms, which may be selected according to the preferred modes of administration. These include, for example, solid, semi-solid and liquid dosage forms such as tablets, pills, powders, liquid solutions or suspensions, suppositories, and injectable and infusible solutions. The preferred form depends on the intended mode of administration and therapeutic application. Modes of administration may include oral, parenteral, subcutaneous, intravenous, intralesional or topical administration.

The enhancing and inhibitory agents of this invention may, for example, be placed into sterile, isotonic formulations with or without cofactors which stimulate uptake or stability. The formulation is preferably liquid (i.e., for oral administration), or may be lyophilized powder. For example, the enhancing and inhibitory agents may be diluted with a formulation buffer comprising 5.0 mg/ml citric acid monohydrate, 2.7 mg/ml trisodium citrate, 41 mg/ml mannitol, 1 mg/ml glycine and 1 mg/ml polysorbate 20. This solution can be lyophilized, stored under refrigeration and reconstituted prior to administration with sterile Water-For-Injection (USP).

The compositions also will preferably include conventional pharmaceutically acceptable carriers well known in the art (see for example Remington's Pharmaceutical Sciences, 16th Edition, 1980, Mac Publishing Company). Such pharmaceutically acceptable carriers may include other medicinal agents, carriers, genetic carriers, adjuvants, excipients, etc., such as human serum albumin or plasma preparations. The compositions are preferably in the form of a unit dose and will usually be administered one or more times a day.

The pharmaceutical compositions of this invention may also be administered using microspheres, liposomes, other microparticulate delivery systems or sustained release formulations placed in, near, or otherwise in communication with affected tissues or the bloodstream. Suitable examples of sustained release carriers include semipermeable polymer matrices in the form of shaped articles such as suppositories or microcapsules. Implantable or microcapsular sustained release matrices include polylactides (U.S. Pat. No. 3,773,319; EP 58,481), copolymers of L-glutamic acid and gamma ethyl-L-glutamate (Sidman et al., Biopolymers, 22, pp. 547-56 (1985)); poly(2-hydroxyethyl-methacrylate) or ethylene vinyl acetate (Langer et al., J. Biomed. Mater. Res., 15, pp. 167-277 (1981); Langer, Chem. Tech., 12, pp. 98-105 (1982)).

Liposomes containing enhancing and inhibitory agents of the invention can be prepared by well-known methods (See, e.g. DE 3,218,121; Epstein et al., Proc. Natl. Acad. Sci. U.S.A., 82, pp. 3688-92 (1985); Hwang et al., Proc. Natl. Acad. Sci. USA., 77, pp. 4030-34 (1980); U.S. Pat. Nos. 4,485,045 and 4,544,545). Ordinarily the liposomes are of the small (about 200-800 Angstroms) unilamellar type in which the lipid content is greater than about 30 mol. % cholesterol. The proportion of cholesterol is selected to control the optimal rate of molecule release.

The enhancing and inhibitory agents molecules of this invention may also be attached to liposomes, which may optionally contain other agents to aid in targeting or administration of the compositions to the desired treatment site. Attachment of enhancing and inhibitory agents to liposomes may be accomplished by any known cross-linking agent such as heterobifunctional cross-linking agents that have been widely used to couple toxins or chemotherapeutic agents to antibodies for targeted delivery. Conjugation to liposomes can also be accomplished using the carbohydrate-directed cross-linking reagent 4-(4-maleimidophenyl)butyric acid hydrazide (MPBH) (Duzgunes et al., J. Cell. Biochem. Abst. Suppl. 16E 77 (1992)).

The following are examples which illustrate the compositions and methods of this invention. These examples should not be construed as limiting: the examples are included for the purposes of illustration only.

### EXAMPLE 1

### Effect of AICARFT Inhibitor on Lipid Accumulation during Adipocyte Differentiation

AICARFT inhibitor CPD-01 was tested in a lipid accumulation adipocyte differentiation assay (see, e.g., Habinowski, S. A. et al., Biophys. Res. Commun. 286, pp. 852-856 (2001)). Briefly, 3T3-L1 cells were plated in complete 10% FBS DMEM media and grown at 37° C. with 5% CO₂ with feeding every two to three days. After six to eight days in culture, complete media was removed and differentiation media (complete media with 4 μg/ml insulin, 0.25 μM dexamethasone, and 0.5 mM 3-isobutyl-1-methylxanthine) was added to separate cell samples with control or test compounds. After three days, the media was changed back to complete media but with the same concentration of control and test compounds. After three to four additional days, the cells were washed in 1×PBS and fixed in 10% paraformaldehyde for 10 minutes at room temperature. The paraformaldehyde was removed and the cells were washed twice with PBS. The lipid accumulated in cells was stained with Oil Red O for 15-30 minutes at room temperature. The stain was removed and the cells were washed three times with 60% isopropanol. Stain was extracted with 4% Igepal in PBS incubated overnight at room temperature with agitation. The supernatant was removed and added to a 96 well plate and the absorbance was read at 492 nm. (FIG. 3)

### EXAMPLE 2

### Effect of an AICARFT Inhibitor on Fatty Acid Oxidation

Compound CPD-01 was tested in our fatty acid oxidation assay. Beta-oxidation was determined by measuring the ¹⁴CO₂ release as described (Harwood, H. J. et al., J. Biol. Chem., 278, pp. 37099-37111 (2003)) with modifications, as follows. Briefly, C2C12 muscle cells or 3T3-L1 differentiated adipocytes were detached from flasks, counted and resuspended in glass flasks with assay buffer (20 mM Hepes, 25 mM NaHCO₃, 1.2 mM KH₂PO₄, 3 mM glucose, 114 mM NaCl, 4.7 mM KCl, 1.2 mM MgSO₄, 2.5 mM CaCl₂, 1% ultra fatty acid-free BSA). After 30 min incubation with assay buffer, 0.5 ml aliquots of assay buffer containing various concentrations of test compounds were added to each flask. Immediately, a solution containing 12% ultra fatty acid-free BSA, L-carnitine and [¹⁴C]-1-palmitate was added to flasks. Flasks were capped with a rubber stopper into which a center well was inserted. After 2 hours incubation, 400 μl of 1.0 M benzethonium was added into center wells and the reaction was terminated with 500 μl of 7% perchloric acid. After incubating the flasks overnight, the center wells were removed. Captured ¹⁴CO₂ was quantitated using a Trilux scintillation counter. (FIGS. 4 and 5)

### EXAMPLE 3

### Effect of AICARFT Inhibitor on Glucose Uptake in 3T3L1 Adipocytes

Differentiated 3T3L1 cells were cultured for 24 hours with or without AICARFT inhibitor CPD-01. Cells were washed twice with DMEM and serum-starved (DMEM/0.1% BSA) for 4 hours in the presence or absence of AICARFT inhibitor CPD-01. Cells were then washed twice with KRH/0.1% BSA. Control wells were treated with insulin or cytochalasin B for 60 min, followed by the addition of a mixture of ³H-2-deoxy-D-glucose (Perkin Elmer) and unlabeled 2-deoxy-D-glucose (Sigma). After a 20 min incubation (37° C.; 5% CO₂) cells were washed 3 times with ice-cold PBS and solubilized with 0.5 M NaOH. Cell-associated radioactivity (proportional to glucose uptake) was assessed by scintillation counting. (FIG. 6)

### EXAMPLE 4

### Effect of AICARFT Inhibitor on Fatty Acid Synthesis in HepG2 Cells

HepG2 cells were seeded at 2×10⁵ cells per well in a 12-well plate. Test compounds (30 μM of CPD-01 and 1 mM of AICAR) were added the second day and incubated with cells overnight. Fatty acid synthesis assays were carried out using an extraction procedure as follows: 1 μCi of ¹⁴C-acetate was added per well and cells were incubated for 2 hours at 37° C. with gentle shaking. The supernatant was removed and 0.5 ml of PBS or 0.5 ml of MicroScint E® organic-based scintillation fluid was added to the cells. Cells were scraped off the plate and transferred to 4 ml scintillation vials. MicroScint-E® (3 ml) was added and the scintillation vials were shaken for 1 hour prior to quantitation by scintillation counting. (FIG. 7)

### EXAMPLE 5

### RNAi Knockdown of ATIC in C2C12 Cells

The day before transfection, 10⁴ C2C12 (mouse muscle myoblast) cells per well were seeded in a 24-well plate with 0.5 ml of culture medium. The following day the cells were transfected with one of three different siRNA duplexes directed to nucleic acid sequences encoding mouse ATIC at a final concentration of 150 nM. The siRNA duplexes were designed and synthesized by Ambion (Austin, Tex.). The siRNA sequences were as follows: siRNA ID 79903, sense sequence 5′-GGGUUCCCUGAAAUGUUAGtt-3′ and antisense sequence 5′-CUAACAUUUCAGGGAACCCtg-3′; siRNA ID 79999, sense sequence 5′-GGCUUGAUUUCAACCUUGUtt-3′ and antisense sequence 5′-ACAAGGUUGAAAUCAAGCCtg-3′; siRNA ID 80093 sense sequence 5′-GGAUUCAUAAACUUGUGUGtt-3′ and antisense sequence 5′-CACACAAGUUUAUGAAUCCtg-3′. A scrambled control siRNA, Silencer™ (Cat # 4611, Ambion) was included as negative control siRNA. Transfection was carried out using RNAiFect® (Qiagen, Valencia, Calif.).

The effect on phospho-AMPK levels was determined by Western blot 72 hours post transfection using phospho-AMPK-alpha (Thr172) (40H9) antibody (Cell Signaling Technology, Beverley, Mass.) according to manufacturer's instructions. Beta-actin was used as an internal loading control. FIG. 8(A).

Gene silencing at the mRNA level was monitored by quantitative RT-PCR 48 hours post transfection using DNA primers specific for AICARFT. Shown in FIG. 8(B) are PCR products using primers specific for AICARFT and 18s rRNA (QuantumRNA™ Classic 18S Internal Standard, Ambion, Austin, Tex.) included as an internal control. RNA was extracted from transfected cells and 400 ng total RNA was subjected to RT-PCR. RT-PCR was carried out at 50° C. for 30 min, followed by 95° C. for 15 min and followed by 24 cycles of 1 min at 94° C., 1 min at 57° C. and 1 min at 72° C.

### EXAMPLE 6

### Assay of AICARFT Activity

AICARFT activity may be assayed, e.g., by the method of Black et al (Analytical Biochem., 90, pp. 397-401 (1978)). Spectrophotometric increase at A298 may be used to monitor AICARFT activity. In addition, radiolabeled substrates are available.

We claim:

1. A method for treating obesity, type 2 diabetes, insulin resistance, metabolic syndrome and syndromes, conditions and complications associated with any of the foregoing, comprising the step of administering to an animal in need thereof an inhibitor of AICARFT in an amount sufficient to inhibit AICARFT enzyme activity.

2. The method of claim 1, wherein the treatment is for obesity.

3. The method of claim 1, wherein the treatment is for type 2 diabetes.

4. The method of claim 1, wherein the treatment is for insulin resistance.

5. The method of claim 1, wherein the treatment is for metabolic syndrome.

6. The method of any one of claims 1-5, wherein the animal is a human.

7. A method for increasing endogenous AICAR-monophosphate levels in a metabolic cell or tissue comprising the step of administering to the cell or tissue an inhibitor of AICARFT in an amount sufficient to increase AMP kinase activity.

8. A method for increasing the oxidation of fatty acids in a metabolic cell or tissue comprising the step of administering to the cell or tissue an inhibitor of AICARFT in an amount sufficient to inhibit AICARFT enzyme activity.

9. A method for increasing glucose uptake in a metabolic cell or tissue comprising the step of administering to the cell or tissue an inhibitor of AICARFT in an amount sufficient to inhibit AICARFT enzyme activity.

10. A method for decreasing fatty acid synthesis in a metabolic cell or tissue comprising the step of administering to the cell or tissue an inhibitor of AICARFT in an amount sufficient to inhibit AICARFT enzyme activity.

11. A method for inhibiting AICARFT in a metabolic cell or tissue comprising the step of administering to the cell or tissue an inhibitor of AICARFT in an amount sufficient to mimic the effect of AICAR on AMP kinase activity when administered at a lower concentration.

12. The method of any one of claims 7-11, wherein the metabolic tissue or cell is selected from the group consisting of muscle, liver, adipose, pancreatic beta cells and cells of the brain that control metabolic homeostasis.

13. The method of any one of claims 1-12, wherein the AICARFT inhibitor binds selectively to AICARFT compared to another folate-dependent enzyme.

14. The method of claim 13, wherein the AICARFT inhibitor binds selectively to AICARFT compared to its binding to DHFR.

15. The method of claim 13, wherein the AICARFT inhibitor binds selectively to AICARFT compared to its binding to GARFT.

16. The method of any one of claims 1-15, wherein the AICARFT inhibitor has an IC₅₀ for AICARFT selected from about: 75 μM-100 μM, 25 μM-50 μM, 5 μM-10 μM, 1 μM, 0.5 μM or 0.1 μM, 50-100 nM, 25-50 nM, 5-25 nM, 1-5 nM, and less than 1 nM.

17. The method of any one of claims 1-6, wherein treatment is selected from IP, IV, oral, transdermal and local administration into muscle or fat.

18. The method of any one of claims 1-6, wherein treatment is oral.

19. The method of any one of claims 1-6, wherein the inhibitor of AICARFT is administered at a does in the range of 0.1 to 1000 mg/kg/BID.

20. The method of any one of claims 1-19, wherein administration of the inhibitor results in at least 5% inhibition of AICARFT activity over a 24 hour period.

21. The method of any one of claims 1-19, wherein administration of the inhibitor results in from about 5% to about 10% inhibition of AICARFT activity over a 24 hour period.

22. The method of any one of claims 1-19, wherein administration of the inhibitor results in from about 10% to about 20% inhibition of AICARFT activity over a 24 hour period.

23. The method of any one of claims 1-19, wherein administration of the inhibitor results in from about 20% to about 50% inhibition of AICARFT activity over a 24 hour period.

24. The method of any one of claims 1-19, wherein administration of the inhibitor results in more than 50% inhibition of AICARFT activity over a 24 hour period.

25. A method for identifying an agent useful for treating obesity, type 2 diabetes, insulin resistance, metabolic syndrome and syndromes, conditions and/or complications associated with any of the foregoing, comprising the step of screening one or more putative agents in a metabolic tissue or cell that modulate one or more of endogenous AICAR-MP levels, AMP kinase activity, fatty acid beta-oxidation, glucose uptake and fatty acid synthesis through AICARFT inhibition, wherein an agent that increases AICAR-MP levels, AMP kinase activity, fatty acid beta-oxidation or glucose uptake, or decreases fatty acid synthesis through AICARFT inhibition is identified as a useful agent.
